(19) United States
(12) Patent Application Publication
(10) Pub. No.: US 2023/0117994 A1
(43) Pub. Date: Apr. 20, 2023

(54) MEDICO-SURGICAL TUBES

(71) Applicant: SMITHS MEDICAL INTERNATIONAL LIMITED, Ashford (GB)

(72) Inventors: Neil Veasey, Ashford (GB); Christopher John Woosnam, London (GB); Andrew Thomas Jeffrey, Marsh (GB); Timothy Bateman, Hythe (GB)

(73) Assignee: SMITHS MEDICAL INTERNATIONAL LIMITED, Ashford (GB)

(21) Appl. No.: 17/913,881

(22) PCT Filed: Mar. 19, 2021
(86) PCT No.: PCT/GB2021/000033

§ 371 Date: Sep. 23, 2022

(30) Foreign Application Priority Data
Apr. 2, 2020 (GB) 2004894.8

(51) Int. Cl. A61M 16/04

(52) U.S. Cl. CPC A61M 16/0465; A61M 2205/0216

(57) ABSTRACT

A tracheostomy tube comprises a skeletal frame (10) of a relatively stiff plastics covered by an overmoulding (11) of a relatively soft, transparent plastics. The frame (10) includes a portion extending along the shaft (1) of the tube and formed by longitudinal struts (16) linked by spaced ribs (20). The shaft portion of the frame (10) is formed integrally with a portion extending along a flange (5) of the tube, which includes apertures (6) for receiving the ends of a neck strap, and apertures (41) for use with sutures. At its patient end (2) the tube has a soft tip (51) formed by an extension of the overmoulding (11) beyond the patient end of the frame (10). At the machine end (3) of the tube the frame (10) is formed with a connector (4).

This invention relates to medico-surgical tubes of the kind having a shaft with a patient end arranged to extend within the body and a flange towards its machine end arranged to lie against the skin surface.

The invention is more particularly, but not exclusively, concerned with tracheostomy tubes.

Tracheostomy tubes are used to enable ventilation or respiration of a patient. They are inserted into the trachea via a surgically formed opening in the neck so that one end of the tube locates in the trachea and the other end locates outside the patient adjacent the neck surface. Tracheostomy tubes are generally used for more long-term ventilation or where it is not possible to insert an airway through the mouth or nose. Various types of different tracheostomy tubes are presently available to suit different needs. The tube may be reinforced along its length, such as with a helically wound metal wire; where there is a risk the tube might be crushed and occluded, such as might happen when the tube is flexed. Alternatively, the tube may be moulded of a soft material, such as a silicone plastics material, where there is a greater risk of trauma to the tracheal lining, such as in paediatric patients or patients with a damaged trachea. It has been proposed to form tracheostomy tubes of a combination of different materials. For example, U.S. Pat. No. 7,918,227 describes an endotracheal tube having a spiral reinforcement and a separate machine end coupling. U.S. Pat. No. 8,783,254 describes a tube where the shaft is formed by a relatively stiff spine and ribs that is covered by a sheath of a softer material. WO15075412 describes a tube where the machine end coupling and a spiral reinforcement are moulded of a relatively stiff material and a softer material is overmoulded to form the shaft and flange.

If the flange is moulded of a soft plastics material it can create problems in that the material may not be strong enough to ensure that the neck strap does not tear the flange where it is threaded through the eyelets used to secure the tube to the patient's neck. In an attempt to reduce this risk, it has been proposed to add an additional strengthening component around the neck strap eyelets as described in U.S. Pat. No. 9,457,164. Existing proposals do not entirely address the problem of providing a tube with a flange that is both soft and conformable yet also has sufficient strength to prevent collapse and to ensure that the flange can be secured safely both by a neck strap and by sutures through the flange.

It is an object of the present invention to provide an alternative medico-surgical tube.

According to one aspect of the present invention there is provided a medico-surgical tube of the above-specified kind, characterised in that the tube includes a skeletal frame of a relatively stiff plastics material extending along both the shaft and the flange, that the tube further includes an overmoulding of a relatively soft plastics material formed on the frame along both the shaft and the flange, that the patient end tip of the shaft is provided by the soft material of the overmoulding, and that the frame in the flange provides an enclosed aperture on opposite sides to receive a neck strap.

The frame may include a connector at the machine end of the tube projecting from the machine side of the flange. The portion of the frame in the flange may be shaped with a plurality of suture openings. The portion of the frame along the shaft may include a plurality of longitudinal struts linked by a plurality of ribs. The material of the overmoulding is preferably transparent or translucent. The frame and overmoulding may be different grades of the same plastics. The thickness of the overmoulding is preferably equal to or greater than the thickness of the frame along the shaft. The thickness of the overmoulding in the flange is preferably greater than the thickness of the overmoulding in the shaft. The overmoulding preferably extends beyond the frame at the patient end of the shaft by a distance between 2 mm and 5 mm. The tube may be a tracheostomy tube.

According to another aspect of the present invention there is provided a method of making a medico-surgical tube comprising moulding a skeletal frame of a relatively stiff plastics material with an integral shaft portion and flange portion, and overmoulding on the frame over both the shaft and flange portion a relatively soft plastics material such that a patient end tip of the tube is provided by the soft material and such that the frame in the flange portion provides an enclosed aperture on opposite sides to receive a neck strap and a further enclosed aperture on opposite sides to receive a suture.

According to a further aspect of the present invention there is provided a tube made by a method according to the above other aspect of the present invention.

A tracheostomy tube and various modifications according to the present invention will now be described, by way of example, in which:

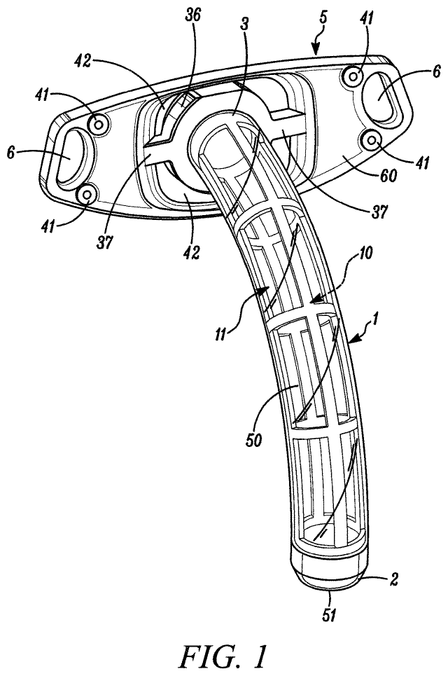

FIG. 1 is a perspective view of the tube;

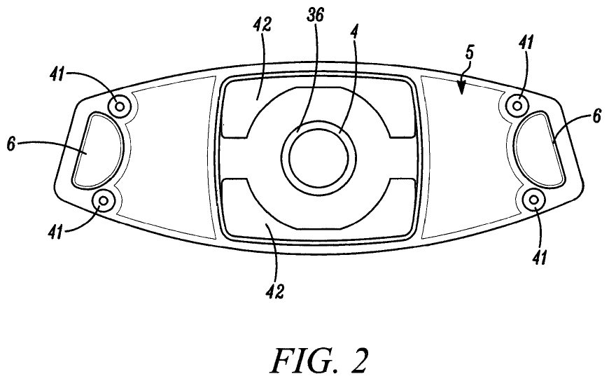

FIG. 2 is an end view of the machine end and flange of the tube;

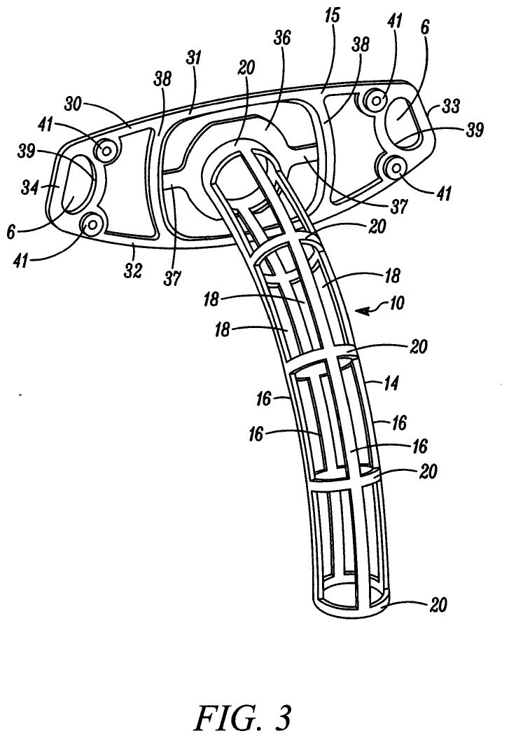

FIG. 3 is a perspective view of the tube at a preliminary stage of manufacture.

With reference first to FIGS. 1 to 2 there is shown a tracheostomy tube having a tubular shaft 1 curved along its length from its patient end 2 to its machine end 3. The machine end 3 of the tube is terminated by a coupling 4 having an external male luer taper and a flange 5 extending radially outwards from the coupling. The flange 5 has enclosed apertures 6 close to opposite ends for use in securing a tape or the like by which the tube is fastened about the patient's neck. The tube is shown without a sealing cuff at its patient end, but it could include a conventional sealing cuff and other conventional features such as a suction lumen for removing secretions that collect above the cuff.

As so far described the tube is conventional. The tube is installed, in use, by inserting its patient end 2 through a surgically-made opening in the neck so that the patient end locates in the trachea with the machine end 3 extending through the neck tissue and with the flange 5 lying adjacent the skin on the outside of the patient.

With reference now also to FIG. 3, the tube differs from previous tubes by being formed from the combination of a skeletal frame 10 and an overmoulding 11. The frame 10 shown in FIG. 3 is moulded of a relatively stiff plastics material, such as a relatively hard grade of silicone or PVC, nylon or ABS. Where the tube is intended to be reusable after reprocessing by autoclave, materials capable of withstanding the autoclave temperature and the temperature used in high temperature silicone vulcanisation could be used, such as PEEK, polysolfone or the like. The overmoulding 11 is formed on the frame 10 and is of a relatively soft plastics material, such as a softer grade of PVC or a silicone material.

The frame 10 consists of a tube section 14 and an integral flange section 15 projecting radially outwardly of the machine end of the tube section. The tube section 14 extends along the shaft 10 and the flange section 15 extends along the flange 5. The flange section 15 also includes the coupling 4.

The tube section 14 comprises at least one longitudinal strut 16. In the present embodiment the tube section 14 comprises four such struts 16 spaced equally from one another with a first strut extending along the outside of the curve of the tube, a second strut extending opposite the first along the inside of the curve, and a third and fourth strut positioned opposite one another along opposite sides of the tube section. The struts 16 are relatively narrow, with the gaps 18 between adjacent struts being wider than the width of the struts. The tube section 14 also comprises several circular rings or ribs 20 extending in a plane oriented radially relative to the longitudinal axis of the section. In the present embodiment the tube section 14 has five such ribs 20 spaced equally along the section. One rib 20 is located at the patient end of the tube section 14, another rib is located at the opposite end adjacent the flange section 15, with the remaining three ribs being equally spaced between these two ribs. The ribs 20 have the same width and thickness as the struts 16 and are moulded integrally as a single piece with the struts.

The flange section 15 of the frame 10 is moulded integrally as a common component with and of the same material as the tube section 14. The flange section 15 has an outer peripheral ring 30 defining the external shape of the flange 5 and having upper and lower lateral edge strips 31 and 32 gently curved along their length to form convex edges to the flange. Opposite ends of the edge strips 31 and 32 are linked by shorter end webs 33 and 34, which are relatively straight and inclined at a small angle to one another. Centrally of the flange section 15 a circular ring 36 with opposite flattened sides projects radially outwardly from the rearmost rib 20 in the tube section 14. The ring 36 is linked by two short strips 37 projecting radially outwardly opposite one another longitudinally of the flange section 15. The outer ends of the two strips 37 are, in turn, joined with the midpoint of two curved cross-pieces 38 extending at right angles to and bridging the two edge strips 31 and 32. At opposite ends of the flange section 15 respective semicircular bands 39 define, with the webs 33 and 34, the two enclosed neck strap apertures 6, which are of a D shape. In the corners between the outside of each band 39 and the edge strips 31 and 32 there are formed two pairs of enclosed suture rings or apertures 41. The topography of the flange section could differ from that described above and shown in the drawings in order to vary its size, flexibility, strength or attachment features.

The tube is completed by the overmoulding 11 of a softer, more flexible plastics material than that forming the frame 10. The overmoulding plastics may be of a soft silicone or PVC and is preferably transparent or translucent so as to enable any contamination, obstruction or component inside the shaft 1 to be seen externally. The overmoulding 11 has a first portion 50 in the form of a sleeve or sheath extending over the tube section 14 of the frame 10. The first portion 50 has a thickness equal to or slightly greater than that of the tube section 14 of the frame 10 so that it forms a smooth surface on both the inside and outside of the frame. The overmoulding 11 in the first portion 50 extends about 2-5 mm beyond the patient end of the frame section 14 so as to form a soft patient end tip 51 to the tube.

A second common or integral portion 60 of the overmoulding 11 continues over selected areas of the flange section 15 of the frame 10. More particularly, the second portion 60 continues over the flange section 15 except for the neck strap apertures 6 and two C-shape regions 42 defined around the central ring 36 between the two horizontal strips 37 and the outer edge strips 31 and 32. The overmoulding portion 60 may extend over the suture apertures 41, since suturing could be made through the soft overmoulded plastics, or these apertures could be left open. The thickness of the material in the flange overmoulding portion 60 is preferably slightly greater than that of the flange section 15 of the frame 10 so that the overmoulded plastics forms a smooth conformable surface on both the forward, patient face of the flange and on its opposite, machine face.

The overmoulding 11 enables the tube to be formed on its external surface and at its tip from soft grades of PVC or silicone so that it is comfortable and atraumatic for the patient. The frame portion 15 in the flange ensures that sutures and the neck strap can be attached with less risk of tearing the material of the flange. The frame portion 14 along the shaft 1 of the tube gives a stable curve and length to the shaft so that inner cannulae can be a matched more closely to the shaft, giving a better fit in the shaft.

By using an overmoulding of a transparent or translucent material through which the frame is visible the frame could be made in different colours to identify different properties of the tube, such as its size. Alternatively, or additionally, the frame in the flange could be printed or embossed with a legend such as to indicate size, manufacturer or other property such as whether or not the tube is fenestrated.

The combination of the softer overmoulded plastics material with the harder plastics frame enables the tube shaft to be given the required mechanical properties in the desired regions without increasing the wall thickness of the shaft. A tube formed entirely of the softer plastics material could be made more resistant to crushing by increasing its wall thickness, but this would result in a narrower bore and hence reduce gas flow and would increase the pressure drop along the tube. The smaller bore diameter could also increase the risk of occlusion from accumulated secretions. The construction of the present invention, by contrast, enables a relatively high ratio of outside diameter to bore diameter. The relatively large internal diameter allows an inner cannula (not shown) with a relatively large diameter to be used. The inner cannula and the softer plastics of the tube shaft could both be thermo softening so that the combination of the inner cannula and outer tube is also thermo softening.

Where the tube has a sealing cuff, the inflation line for the cuff could be retained by clips formed along a longitudinal member of the shaft portion of the frame.

The arrangement is not confined to tracheostomy tubes but could be used in tubes for other purposes.

1-13. (canceled)

14. A medico-surgical tube having a shaft with a patient end arranged to extend within the body and a flange towards its machine end arranged to lie against the skin surface, characterised in that the tube includes a skeletal frame of a relatively stiff plastics material extending along both the shaft and the flange, that the tube further includes an overmoulding of a relatively soft plastics material formed on the frame along both the shaft and the flange, that the patient end tip of the shaft is provided by the soft material of the overmoulding, and that the frame in the flange provides an enclosed aperture on opposite sides to receive a neck strap.

15. The tube according to claim 14, characterised in that the frame includes a connector at the machine end of the tube projecting from the machine side of the flange.

16. The tube according to claim 14, characterised in that the portion of the frame in the flange is shaped with a plurality of suture openings.

17. The tube according to any claim 14, characterised in that the portion of the frame along the shaft includes a plurality of longitudinal struts linked by a plurality of ribs.

18. The tube according to claim 14, characterised in that the material of the overmoulding is transparent or translucent.

19. The tube according to claim 14, characterised in that the frame and overmoulding are of different grades of the same plastics.

20. The tube according to claim 14, characterised in that the thickness of the overmoulding is equal to or greater than the thickness of the frame along the shaft.

21. The tube according to claim 14, characterised in that the thickness of the overmoulding in the flange is greater than the thickness of the overmoulding in the shaft.

22. The tube according to claim 14, characterised in that the overmoulding extends beyond the frame at the patient end of the shaft by a distance between 2 mm and 5 mm.

23. The tube according to claim 14, wherein the tube is a tracheostomy tube.

24. A method of making a medico-surgical tube including: moulding a skeletal frame of a relatively stiff plastics material with an integral shaft portion and flange portion, and overmoulding on the frame over both the shaft and flange portion a relatively soft plastics material such that a patient end tip of the tube is provided by the relatively soft material and such that the frame in the flange portion provides an enclosed aperture on opposite sides to receive a neck strap.

25. A tracheostomy tube made by the method according to claim 24.
